(19) United States
(12) Patent Application Publication
(10) Pub. No.: US 2023/0064020 A1
(43) Pub. Date: Mar. 2, 2023

(54) POWER SOURCE LONGEVITY IMPROVEMENT FOR A DEVICE

(71) Applicant: Medtronic, Inc., Minneapolis, MN (US)

(72) Inventors: Matthew J. Hoffman, St. Paul, MN (US); Ryan D. Wyszynski, Oak Grove, MN (US); Erin N. Reisfeld, Minneapolis, MN (US); Bo Zhang, Blaine, MN (US); Christopher D. Koch, Minneapolis, MN (US)

(21) Appl. No.: 17/821,675

(22) Filed: Aug. 23, 2022

Related U.S. Application Data
(60) Provisional application No. 63/236,568, filed on Aug. 24, 2021.

(51) Int. Cl. G16H 40/67

(52) U.S. Cl. CPC G16H 40/67

(57) ABSTRACT

This disclosure describes systems, devices and techniques for improving the longevity of battery life in a device. An example first device includes communication circuitry configured to communicate with a second device and processing circuitry configured to determine an expected amount of data to be transmitted by the second device to the first device. The processing circuitry is configured to determine that the expected amount of data to be transmitted by the second device to the first device is greater than or equal to a predetermined data threshold and based on the expected amount of data to be transmitted being greater than or equal to the predetermined data threshold, determine that a predetermined restriction is met. The processing circuitry is configured to, based on the predetermined restriction being met, control the communication circuitry to transmit an instruction to the second device.

This application claims priority to U.S. Provisional Application No. 63/236,568, filed Aug. 24, 2021, the entire content of which is incorporated herein by reference.

### TECHNICAL FIELD

The disclosure relates generally to devices and device systems and, more particularly, to improving longevity of power sources for devices or improving the probability of successful communication between devices, such as medical devices.

### BACKGROUND

Some types of medical devices may be used to monitor one or more physiological parameters of a patient. In addition to or instead of monitoring one or more physiological parameters of a patient, some medical devices may be used to provide therapy to a patient. Such medical devices may include, or may be part of a system that includes, sensors that detect signals associated with physiological parameters. Values determined based on such signals may be used to assist in detecting changes in patient conditions, in evaluating the efficacy of a therapy, or in generally evaluating patient health. Such medical devices may be implantable or external to the patient and be powered by a battery.

### SUMMARY

In general, the disclosure describes techniques for improving the longevity of power sources for devices or increasing the likelihood of successful communication between devices. These techniques may be applicable to external devices or implantable medical devices (IMDs). For example, the techniques described herein may extend a battery life of a battery powering a device or increase a likelihood of successful communications between devices. While the techniques of this disclosure are primarily described with respect to IMDs and external devices, the techniques may be used with any devices powered by a power source, such as a battery.

Because the IMD is implanted within the patient, a clinician or a patient uses an external device to configure or control the monitoring and/or therapy provided by the IMD over a wireless connection. These external devices may also be referred to as programmers or monitors. One type of external device which may be used with an IMD is mobile device, such as a cellular phone (e.g., a smart phone), a satellite phone, a tablet, a wearable device, or the like. Other types of external devices may include devices that are intended to remain stationary, such as a dedicated bed-side monitor, a desktop computer, a server, or the like.

An IMD may wirelessly advertise for communication to the external device at predetermined intervals. The external device may initiate communication with the IMD in response to receiving an advertisement. The external device may then transmit one or more instructions to the IMD. For example, the external device may transmit an instruction for the IMD to transmit data to the external device. When the IMD is transmitting data to the external device, the power source of the IMD (e.g., a battery) is being drained by the wireless radio within the IMD. Some IMDs contain limited and fixed capacity, non-rechargeable batteries, while other IMDs contain rechargeable batteries. It may be desirable to improve the likelihood that any such communication may be successful with either type of IMD. For example, a successful communication may be one in which all data intended to be exchanged during the communication session is exchanged. Improving the likelihood that a communication is successful may reduce the number of times the same data is transmitted by the IMD. For an IMD having a non-rechargeable battery, improving the likelihood that a communication is successful may extend the overall life of the IMD which may reduce a need for surgery to replace the IMD. Improving the likelihood that the communication is successful may also decrease the likelihood that received data is corrupted and may potentially increase the speed at which data is transferred. For an IMD having a rechargeable battery, improving the likelihood that a communication is successful may extend the recharge interval leading to increased patient satisfaction and flexibility.

For example, the external device may be configured to prevent data transmissions of a significant size prior to having a relatively higher probability of completing the transmission. For example, the external device may be configured to prevent data transmissions of a larger than a predetermined size when processing circuitry of the external device determines that there is a relatively low probability of completing the transmission.

In some examples, a first device includes communication circuitry configured to communicate with a second device; and processing circuitry configured to: determine an expected amount of data to be transmitted by the second device to the first device; determine that the expected amount of data to be transmitted by the second device to the first device is greater than or equal to a predetermined data threshold; based on the expected amount of data to be transmitted being greater than or equal to the predetermined data threshold, determine that a predetermined restriction is met; and based on the predetermined restriction being met, control the communication circuitry to transmit an instruction to the second device.

In some examples, a method includes determining, by processing circuitry of a first device, an expected amount of data to be transmitted by a second device to the first device; determining, by the processing circuitry, that the expected amount of data to be transmitted by the second device to the first device is greater than or equal to a predetermined data threshold; determining, by the processing circuitry and based on the expected amount of data to be transmitted being greater than or equal to the predetermined data threshold, that a predetermined restriction is met; and controlling communication circuitry, by the processing circuitry and based on the predetermined restriction being met, to transmit an instruction to the second device.

In some examples, a non-transitory computer-readable medium includes instructions for causing one or more processors to: determine an expected amount of data to be transmitted by a second device to a first device; determine that the expected amount of data to be transmitted by the second device to the first device is greater than or equal to a predetermined data threshold; determine, based on the expected amount of data to be transmitted being greater than or equal to the predetermined data threshold, that a predetermined restriction is met; and control communication circuitry, based on the predetermined restriction being met, to transmit an instruction to the second device.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF DRAWINGS

FIG. 1 illustrates the environment of an example medical device system in conjunction with a patient, in accordance with one or more techniques of this disclosure.

FIG. 2 is a conceptual drawing illustrating an example configuration of the implantable medical device (IMD) of the medical device system of FIG. 1, in accordance with one or more techniques described herein.

FIG. 3 is a functional block diagram illustrating an example configuration of the IMD of FIGS. 1 and 2, in accordance with one or more techniques described herein.

FIGS. 4A and 4B illustrate two additional example IMDs that may be substantially similar to the IMD of FIGS. 1-3, but which may include one or more additional features, in accordance with one or more techniques described herein.

FIG. 5 is a block diagram illustrating an example configuration of components of the external device of FIG. 1, in accordance with one or more techniques of this disclosure.

FIG. 6 is a flow diagram illustrating an example operation for improving power consumption of an IMD, in accordance with one or more techniques of this disclosure.

Like reference characters denote like elements throughout the description and figures.

### DETAILED DESCRIPTION

Various medical devices, including implantable medical devices (IMDs) such as insertable cardiac monitors, pacemakers, cardioverter-defibrillators, cardiac resynchronization devices, left ventricular assist device (LVAD), pulmonary artery pressure sensors, neurostimulators, spinal cord stimulators, drug pumps and other IMDs or wearable medical devices, and devices such as smart phones, blood pressure devices, scales to measure weight, hearing aids, pulse oximeters, cardiac monitoring patches, smart watches, fitness trackers, and other wearable devices, may include sensors which may sense vital physiological parameters of a patient and/or circuitry to provide therapy to the patient. Such medical devices may be configured to communicate with external computing devices through secure wireless communications technologies, such as personal area networking technologies like Bluetooth® or Bluetooth® low energy (BLE) wireless protocol. For example, a patient having such a medical device may be able to transmit and/or receive information relating to the operation of the IMD or to physiological parameters sensed by the IMD via such secure wireless communications technologies through the use of an external device. In some examples, the external device may be a mobile device, such as a cellular phone (e.g., a smart phone), a satellite phone, a tablet, a wearable device (e.g., a smart watch), a laptop computer, or the like. In other examples, the external device may be a more stationary device, such as a desktop computer, a dedicated bed-side monitor, a server, or the like.

In the event that relatively large amounts of data will be transmitted between an IMD and an external computing device, there is an increased risk of a connection drop-out when the patient is ambulatory (due to environmental considerations, proximity to the external computing device, etc.). These increased connection drop-outs can be costly from a battery longevity standpoint, as the IMD may be required to retransmit the same data using an energy-intensive radio frequency communications module. Additionally, data transmitted when a communication link may be unreliable may be more likely to result in data received by the IMD or the external device being corrupted. Therefore, it may be beneficial to transmit data between the IMD and the external device at times when the risk of a connection drop-out is relatively lower. In some examples, if the communication is relatively urgent, the IMD may transmit the data even though there may be a relatively higher risk of a drop-put.

The IMD may periodically, at a regular cadence advertise, e.g., transmit a BLE advertisement, to the external device to begin a communications session if desired. For example, the patient or a clinician, using the external device, may want to query the IMD for physiological parameters sensed by the IMD or to program the IMD. For example, the IMD may have data available for the external device to retrieve. The external device may scan for this advertisement. When desired, the external device may initiate a communication session with the IMD, in response to the advertisement.

Mobile external devices may have environment-dependent connection issues which may be mitigated by optimizing the time of the day/day of the week when, or location where, transmissions occur. Relatively stationary external devices (including bedside monitors) may constantly scan for communication advertisements which may enable them to have a higher probability of discovering the IMD compared to mobile external devices. However, unlike a mobile external device, such as a smart phone, which is often carried with a patient when the patient is ambulatory, a bedside monitor is likely to be stationary relative to the patient. If the IMD inside the patient were to connect to the bedside monitor and then the patient were to walk out of communication range, the connection would time-out, thereby necessitating a second attempt to transmit the data within the IMD to the bedside monitor. The techniques described herein may be used to reduce the number of unsuccessful data transmissions from an IMD to an external device and thereby lengthen the life of a power source of the IMD.

The IMD may have limited battery resources that support both medical activities (e.g., monitoring physiological parameters of a patient, pacing a patient’s heart, delivering stimulation to a nerve of the patient, etc.), as well as the communications requirements with the external device. However, in a typical deployment, a patient having the IMD may move with respect to the external device during a communication session which may cause the communication session to time out or cause disruptions in the exchange of data between the external device and the IMD. For example, a patient having the IMD may leave the external device (e.g., their smart phone) in the car during a communication session, and walk into their home, which is out of communication range of the external device. In scenarios like this, the communication session my time out and have to be reinitiated and data re-exchanged. This is wasteful of power from the battery of the IMD. Therefore, it may be desirable to preserve battery capacity by placing restrictions on when the external device and the IMD may initiate a communication session. Such restrictions may increase the likelihood of successful communications between the IMD and the external device, e.g., where all data intended to be exchanged during a given communication session is exchanged. Preserving battery capacity may extend the life of the IMD or increase the recharge interval of the IMD, either of which may be desirable to the patient.

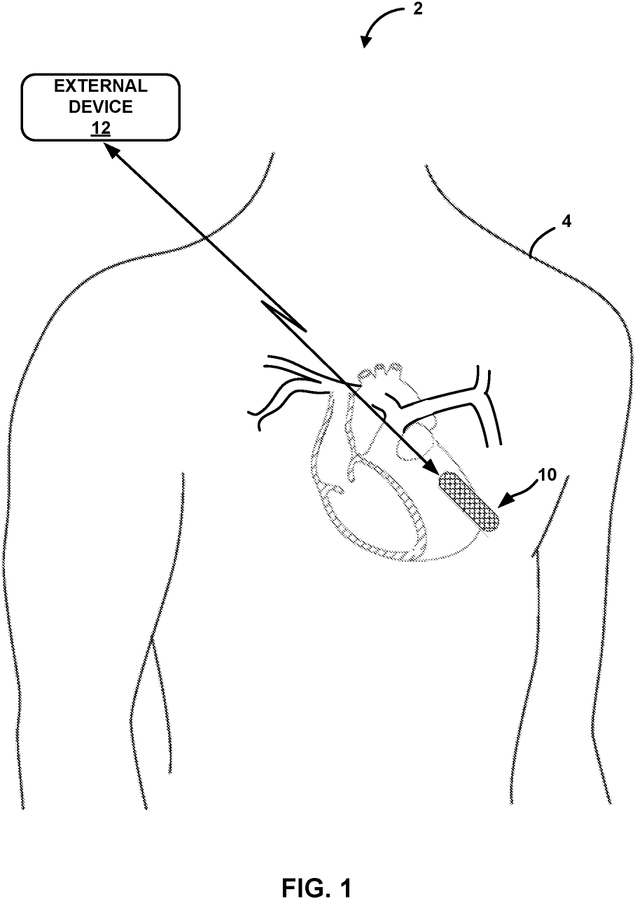

FIG. 1 illustrates the environment of an example medical device system 2 in conjunction with a patient 4, in accordance with one or more techniques of this disclosure. The example techniques may be used with IMD 10, which may be in wireless communication with external device 12. In some examples, IMD 10 is implanted outside of a thoracic cavity of patient 4 (e.g., subcutaneously in the pectoral location illustrated in FIG. 1). IMD 10 may be positioned near the sternum near or just below the level of patient 4’s heart, e.g., at least partially within the cardiac silhouette. In some examples, IMD 10 takes the form of a LINQ™ Insertable Cardiac Monitor (ICM), available from Medtronic plc, of Dublin, Ireland. The example techniques may additionally, or alternatively, be used with a medical device not illustrated in FIG. 1, such as another type of IMD or an external medical device. For example such techniques may be used with a diabetes pump, drug pump, or the like.

Although in one example IMD 10 takes the form of an ICM, in other examples, IMD 10 takes the form of any combination of implantable cardioverter defibrillators (ICDs) with intravascular or extravascular leads, pacemakers, cardiac resynchronization therapy devices (CRT-Ds), neuromodulation devices, left ventricular assist devices (LVADs), implantable sensors, cardiac resynchronization therapy pacemakers (CRT-Ps), implantable pulse generators (IPGs), orthopedic devices, drug pumps, or other IMDs as examples. Moreover, techniques of this disclosure may be used reduce the battery drain of one or more of the aforementioned devices.

Clinicians sometimes diagnose a patient (e.g., patient 4) with medical conditions and/or determine whether a condition of patient 4 is improving or worsening based on one or more observed physiological signals collected by physiological sensors, such as electrodes, optical sensors, chemical sensors, temperature sensors, acoustic sensors, and motion sensors. In some cases, clinicians apply non-invasive sensors to patients in order to sense one or more physiological signals while a patent is in a clinic for a medical appointment. However, in some examples, events that may change a condition of a patient, such as administration of a therapy, may occur outside of the clinic. As such, in these examples, a clinician may be unable to observe the physiological markers needed to determine whether an event has changed a medical condition of the patient and/or determine whether a medical condition of the patient is improving or worsening while monitoring one or more physiological signals of the patient during a medical appointment. In the example illustrated in FIG. 1, IMD 10 is implanted within patient 4 to continuously record one or more physiological signals of patient 4 over an extended period of time.

In some examples, IMD 10 includes a plurality of electrodes. The plurality of electrodes is configured to detect signals that enable processing circuitry of IMD 10 to determine current values of additional parameters associated with the cardiac and/or lung functions of patient 4. In some examples, the plurality of electrodes of IMD 10 are configured to detect a signal indicative of an electric potential of the tissue surrounding the IMD 10. Moreover, IMD 10 may additionally or alternatively include one or more optical sensors, accelerometers, temperature sensors, chemical sensors, light sensors, pressure sensors, and acoustic sensors, in some examples. Such sensors may detect one or more physiological parameters indicative of a patient condition.

In some examples, external device 12 may be a hand-held computing device with a display viewable by the user and an interface for providing input to external device 12 (e.g., a user input mechanism). For example, external device 12 may include a small display screen (e.g., a liquid crystal display (LCD) or a light emitting diode (LED) display) that presents information to the user. In addition, external device 12 may include a touch screen display, keypad, buttons, a peripheral pointing device, voice activation, or another input mechanism that allows the user to navigate through the user interface of external device 12 and provide input. If external device 12 includes buttons and a keypad, the buttons may be dedicated to performing a certain function, e.g., a power button, the buttons and the keypad may be soft keys that change in function depending upon the section of the user interface currently viewed by the user, or any combination thereof. In some examples, external device 12 may be a mobile device, such as a cellular phone (e.g., a smart phone), a satellite phone, a tablet, a laptop computer, or a wearable device (e.g., a smart watch). In some examples, external device 12 may be a relatively stationary device, such as a desktop computer, a server, or a dedicated bedside monitor.

When external device 12 is configured for use by the clinician, external device 12 may be used to transmit instructions to IMD 10. Example instructions may include requests to set electrode combinations for sensing and any other information that may be useful for programming into IMD 10. The clinician may also configure and store operational parameters for IMD 10 within IMD 10 with the aid of external device 12. In some examples, external device 12 assists the clinician in the configuration of IMD 10 by providing a system for identifying potentially beneficial operational parameter values.

Whether external device 12 is configured for clinician or patient use, external device 12 is configured to communicate with IMD 10 and, optionally, another computing device (not illustrated by FIG. 1), via wireless communication. External device 12, for example, may communicate via near-field communication technologies (e.g., inductive coupling, NFC or other communication technologies operable at ranges less than 10-20 cm) and far-field communication technologies (e.g., RF telemetry according to the 802.11 or Bluetooth®, BLE specification sets, or other communication technologies operable at ranges greater than near-field communication technologies). In some examples, external device 12 is configured to communicate with a computer network, such as the Medtronic CareLink® Network developed by Medtronic, plc, of Dublin, Ireland. For example, external device 12 may transmit data, such as data received from IMD 10, to another external device such as a smartphone, a tablet, or a desktop computer, and the other external device may in turn transmit the data to the computer network. In other examples, external device 12 may directly communicate with the computer network without an intermediary device.

Medical device system 2 of FIG. 1 is an example of a system configured to collect an electrogram (EGM) signal according to one or more techniques of this disclosure. In some examples, processing circuitry 14 includes EGM analysis circuitry configured to determine one or more parameters of an EGM signal of patient 4. In one example, an EGM signal is sensed via one or more electrodes of IMD 10. An EGM is a signal representative of electrical activity of the heart, measured by electrodes implanted within the body, and often within the heart itself. For example, a cardiac EGM may include P-waves (depolarization of the atria), R-waves (depolarization of the ventricles), and T-waves (repolarization of the ventricles), among other events. Information relating to the aforementioned events, such as time separating one or more of the events, may be applied for a number of purposes, such as to determine whether an arrhythmia is occurring and/or predict whether an arrhythmia is likely to occur. Cardiac signal analysis circuitry, which may be implemented as part of processing circuitry 14, may perform signal processing techniques to extract information indicating the one or more parameters of the cardiac signal.

In some examples, IMD 10 includes one or more accelerometers. An accelerometer of IMD 10 may collect an accelerometer signal which reflects a measurement of any one or more of a motion of patient 4, a posture of patient 4 and a body angle of patient 4. In some cases, the accelerometer may collect a three-axis accelerometer signal indicative of patient 4’s movements within a three-dimensional Cartesian space. For example, the accelerometer signal may include a vertical axis accelerometer signal vector, a lateral axis accelerometer signal vector, and a frontal axis accelerometer signal vector. The vertical axis accelerometer signal vector may represent an acceleration of patient 4 along a vertical axis, the lateral axis accelerometer signal vector may represent an acceleration of patient 4 along a lateral axis, and the frontal axis accelerometer signal vector may represent an acceleration of patient 4 along a frontal axis. In some cases, the vertical axis substantially extends along a torso of patient 4 when patient 4 from a neck of patient 4 to a waist of patient 4, the lateral axis extends across a chest of patient 4 perpendicular to the vertical axis, and the frontal axis extends outward from and through the chest of patient 4, the frontal axis being perpendicular to the vertical axis and the lateral axis.

IMD 10 may measure a set of parameters including an impedance (e.g., subcutaneous impedance, an intrathoracic impedance or an intracardiac impedance) of patient 4, a respiratory rate of patient 4 during night hours, a respiratory rate of patient 4 during day hours, a heart rate of patient 4 during night hours, a heart rate of patient 4 during day hours, an atrial fibrillation (AF) burden of patient 4, a ventricular rate of patient 4 while patient 4 is experiencing AF, or any combination thereof.

In some examples, one or more sensors (e.g., electrodes, motion sensors, optical sensors, temperature sensors, or any combination thereof) of IMD 10 may generate a signal that indicates a physiological parameter of a patient. In some examples, the signal that indicates the physiological parameter includes a plurality of parameter values, where each parameter value of the plurality of parameter values represents a measurement of the parameter at a respective interval of time. The plurality of parameter values may represent a sequence of parameter values, where each parameter value of the sequence of parameter values are collected by IMD 10 at a start of each time interval of a sequence of time intervals. For example, IMD 10 may perform a parameter measurement in order to determine a parameter value of the sequence of parameter values according to a recurring time interval (e.g., every day, every night, every other day, every twelve hours, every hour, or any other recurring time interval). In this way, IMD 10 may be configured to track a respective patient parameter more effectively as compared with a technique in which a patient parameter is tracked during patient visits to a clinic, since IMD 10 is implanted within patient 4 and is configured to perform parameter measurements according to recurring time intervals without missing a time interval or performing a parameter measurement off schedule.

As discussed above, IMD 10 may have limited battery resources and during transmission of larger amounts of data from IMD 10 to external device 12, there may be a higher likelihood that the communication session is interrupted either due to environmental effects or from patient 4 moving away from external device 12. If the communication session between external device 12 and IMD 10 is interrupted, such as times out, any data meant to be transferred between external device 12 and IMD 10 may have to be retransmitted. This places a burden on the battery of IMD 10. The retransmitting of data may shorten the life of an IMD having a non-rechargeable battery and shorten the recharge interval of an IMD having a rechargeable battery, neither of which is desirable.

As such, according to the techniques of this disclosure, external device 12 may determine an expected amount of data to be transmitted by IMD 10 to external device 12 and place one or more restrictions on when data transmissions occur between external device 12 and IMD 10, for example, when the expected amount of data to be transmitted is greater than or equal to a predetermined data threshold. For example, external device 12 may have some insight into the amount of data to be transmitted based on the type of instruction external device may transmit to IMD 10 (e.g., an instruction to transmit all stored physiological data, an instruction to transmit the last hour’s stored physiological data, etc.), the last time IMD 10 transmitted stored physiological data to external device 12, and/or the amount of data that has been transmitted by IMD 10 to external device 12 in the past.

In another example, an advertisement for communication transmitted by IMD 10 may include a universally unique identifier (UUID) which may be indicative of a payload size that IMD 10 may transmit or a relative urgency of a transmission. For example, UUID1 may indicate a payload of between 0 and 1 kb, UUID2 may indicate a payload of between 1 kb and 10 kb, UUID3 may indicate a payload of between 10 kb and 100 kb, and so on. In another example, UUID1 may indicate a low-urgency transmission, UUID2 may indicate a medium-urgency transmission, UUID3 may indicate a high-urgency transmission, and so on. In another example, the time of day may be indicative of the size of the payload to be transmitted. For example, between 12 am and 6 am the payload may be between 0 and 100 kb and between 6 am and 12 am the payload may be greater than 100 kb.

In another example, IMD 10 may include information within the advertisement such as the urgency of the data to be transmitted, the size of the payload, or the like. External device 12 may use such information to determine whether to connect to IMD 10 or not.

In another example, IMD 10 may transmit an expected payload size relatively early in a communication with external device 12 and external device 12 may use such expected payload size to determine whether to continue or terminate the communication session.

In some examples, IMD 10 may change the time period between advertising intervals based on type of data to be transmitted to external device 12 and/or the size of the payload. For example, when IMD 10 has a relatively small payload to transmit or data related to a critical event, such as detected ventricular tachycardia, ventricular fibrillation, myocardial infarction, or the like, IMD 10 may decrease the time period between advertising intervals. When IMD 10 has less important data to transmit or a relatively large payload, IMD 10 may increase the time period between advertising intervals.

As mentioned above, external device 12 may determine an expected amount of data to be transmitted by IMD 10 to external device 12. For example, external device 12 may determine that the expected amount of data to be transmitted by IMD 10 to external device 12 is less than a predetermined data threshold in which case external device 12 may transmit an instruction to IMD 10 as a relatively smaller amount of data may be less likely to have to be retransmitted than a larger amount of data and, if the smaller amount of data had to be retransmitted, it would be less power intensive than retransmitting a larger amount of data.

For example, external device 12 may determine that the expected amount of data to be transmitted by IMD 10 to external device 12 is greater than or equal to a predetermined data threshold. External device 12 may, based on the expected amount of data to be transmitted being greater than or equal to the predetermined data threshold, determine that a predetermined restriction is met. External device 12 may, based on the predetermined restriction being met, control communication circuitry to transmit an instruction to the implanted medical device. This instruction may be an instruction for IMD 10 to transmit the data to be transmitted to external device 12. In this manner, external device 12 may control when IMD 10 transmits relatively large amounts of data so as to reduce the likelihood that the transmission is interrupted, and thereby save battery power by reducing retransmissions of the data.

In another example, external device 12 may, based on the expected amount of data to be transmitted being greater than or equal to the predetermined data threshold, determine that a predetermined restriction is not met. External device 12 may, based on the predetermined restriction not being met, control communication circuitry to refrain from transmitting an instruction to the implanted medical device. For example, external device 12 may determine that successful communication from IMD 10 to external device 12 is unlikely and wait until a better time for transmitting the instruction or prompt patient 4 to notify external device 12 when patient 4 believes it to be a better time for transmitting the instruction. In some examples, external device 12 may determine that a successful communication from IMD 10 to external device 12 is likely, and based on the determination that a successful communication from IMD 10 to external device 12 is likely, external device may prompt patient 4 (e.g., if patient 4 is ambulatory) to remain in communication range of external device 12 until the communication is complete.

In some examples, external device 12 may implement the techniques of this disclosure in response to a charge level of a battery of IMD 10 or a battery charge level of external device 12 falling below a predetermined charge threshold level. In this manner, external device 12 may not determine an expected amount of data to be transmitted until external device 12 receives an indication from IMD 10 that its battery charge level is below the predetermined battery charge threshold level or determines that the battery charge level of external device 12 is below the predetermined battery threshold level. In some examples, there may be different predetermined battery threshold levels for IMD 10 and for external device 12.

Several examples of potential predetermined restrictions are now discussed. These predetermined restrictions may be used separately, or in any combination.

In some examples, the predetermined restriction includes external device 12 discovering an advertisement for communication from IMD 10 in a predetermined number of consecutive advertising intervals. For example, as discussed above, IMD 10 may transmit advertisements for communication to external device 12 at a time interval known to external device 12. External device 12 may refrain from transmitting the instruction to IMD 10 until external device 12 discovers a predetermined number of consecutive advertisements. For example, if external device 12 knows that IMD 10 transmits an advertisement for communication every minute, external device 12 may wait until external device 12 discovers three advertisements in three minutes (e.g., three consecutive advertisements) prior to transmitting the instruction to IMD 10. In some examples, there may be a plurality of such predetermined numbers of consecutive advertising intervals, each associated with a different predetermined data threshold. For example, external device 12 may require discoveries on two consecutive sequential advertising intervals for a data transmission greater than a first predetermined data threshold and three consecutive sequential advertising intervals for a data transmission of an expected amount of data greater than a second predetermined data threshold, wherein the second predetermined data threshold is greater than the first predetermined data threshold. In some examples, there may be any number of predetermined numbers of consecutive advertising intervals associated with a different predetermined data thresholds. This restriction may reduce the likelihood that the transmission of the data from IMD 10 to external device 12 is interrupted.

In some examples, the predetermined restriction includes external device 12 discovering an advertisement in a first predetermined number of advertising intervals for communication from IMD 10 out of a second predetermined number of consecutive advertising intervals (e.g., an x of y probabilistic restriction). For example, external device 12 may refrain from transmitting the instruction to IMD 10 until external device 12 discovers an advertisement for communication from IMD 10 in a first predetermined number of advertising intervals out of a second predetermined number of consecutive advertising intervals. For example, external device 12 may wait until external device 12 discovers advertisements in three out of four advertisement intervals sent by IMD 10 before transmitting the instruction to IMD 10. In some examples, there may be a plurality of such predetermined numbers of advertising intervals and predetermined number of consecutive advertising intervals, each associated with a different predetermined data threshold. For example, external device 12 may require discoveries in three out of four consecutive advertising intervals for a data transmission greater than a first predetermined data threshold and in four out of five consecutive advertising intervals for a data transmission of greater than a second predetermined data threshold, wherein the second predetermined data threshold is greater than the first predetermined data threshold. In some examples, there may be any number of predetermined probabilistic advertising interval discoveries associated with a different predetermined threshold size of transmission. This restriction may reduce the likelihood that the transmission of the data from IMD 10 to external device 12 is interrupted.

In some examples, the predetermined restriction includes external device 12 discovering a predetermined number of advertisements for communication from IMD 10 during a predetermined period of time. For example, external device 12 may refrain from transmitting the instruction to IMD 10 until external device 12 discovers a predetermined number of advertisements for communication from IMD 10 within a predetermined period of time. For example, external device 12 may wait until external device 12 discovers five advertisements sent by IMD 10 within 15 minutes before transmitting the instruction to IMD 10. In some examples, there may be a plurality of such predetermined numbers of advertisements and predetermined periods of time, each associated with a different predetermined data threshold. For example, external device 12 may require discoveries of three advertisements within fifteen minutes for a data transmission greater than a first predetermined data threshold and four advertisements within ten minutes for a data transmission of greater than a second predetermined data threshold, wherein the second predetermined data threshold is greater than the first predetermined data threshold. In some examples, there may be any number of predetermined number of advertisements and associated predetermined time periods with a different predetermined threshold size of transmission. This restriction may reduce the likelihood that the transmission of the data from IMD 10 to external device 12 is interrupted.

In some examples, the predetermined restriction includes a signal strength of an advertisement for communication from the implantable medical device being greater than or equal to a predetermined signal strength threshold. For example, the predetermined signal strength threshold may be a received signal strength indicator (RSSI) of at least -120 dBm, -100 dBm, or other signal strength. For example, external device 12 may refrain from transmitting the instruction to IMD 10 until external device 12 determines that the signal strength of an advertisement for communication is greater than or equal to the predetermined signal strength threshold. For example, external device 12 may wait until external device 12 determines that the signal strength of an advertisement for communication is greater than or equal to the predetermined signal strength threshold before transmitting the instruction to IMD 10. In some examples, there may be a plurality of such predetermined signal strength thresholds, each associated with a different predetermined data threshold. For example, external device 12 and/or IMD 10 may require an RSSI exceed -90 dBm for a data transmission greater than a first predetermined data threshold and an RSSI exceed -80 dBm for a data transmission of greater than a second predetermined data threshold, wherein the second predetermined data threshold is greater than the first predetermined data threshold. In some examples, there may be any number of predetermined signal strength thresholds associated with different predetermined threshold size of transmission. This restriction may reduce the likelihood that the transmission of the data from IMD 10 to external device 12 is interrupted.

In some examples, the predetermined restriction includes a signal strength of an idle communication link between IMD 10 and external device 12 being greater than or equal to a predetermined signal strength threshold for a predetermined length of time. An idle communication link may be an established communication link where only data needed to keep the communication link up is transmitted, and payload data is not exchanged. In some examples, external device 12 may be configured to control the communication circuitry to transmit the instruction after the predetermined length of time. For example, the predetermined signal strength threshold may be a received signal strength indicator (RSSI) of at least -120 dBm, -100 dBm, or other signal strength. For example, external device 12 may refrain from transmitting the instruction to IMD 10 until external device 12 determines that the signal strength of the idle communication link is greater than or equal to the predetermined signal strength threshold for the predetermined length of time, e.g., three seconds. For example, external device 12 may initiate a communication session with IMD 10 in response to receiving one or more advertisements for communication, but may wait until external device 12 determines that the signal strength of the communication link between external device 12 and IMD 10 is greater than or equal to the predetermined signal strength threshold for the predetermined length of time before transmitting the instruction to IMD 10. In some examples, there may be a plurality of such predetermined signal strength thresholds, each associated with a different predetermined data threshold or there may be a plurality of predetermined lengths of time. For example, external device 12 may require each of the determined RSSIs exceed -90 dBm for a data transmission greater than a first predetermined data threshold and that each of the determined RSSIs exceed -80 dBm for a data transmission of greater than a second predetermined data threshold, wherein the second predetermined data threshold is greater than the first predetermined data threshold. Alternatively, or in addition, the predetermined length of time (e.g., two seconds) for the first predetermined data threshold may be shorter (e.g., fewer seconds) than the predetermined time period (e.g., three seconds) for the second predetermined data threshold. In some examples, there may be any number of predetermined signal strength thresholds and/or predetermined lengths of time associated with different predetermined data thresholds. This restriction may reduce the likelihood that the transmission of the data from IMD 10 to external device 12 is interrupted.

In some examples, the predetermined restriction includes a range of signal strengths of an idle communication link between IMD 10 and external device 12 being smaller than to a predetermined signal strength range threshold for a predetermined length of time. In some examples, external device 12 is configured to control the communication circuitry to transmit the instruction after the predetermined length of time. For example, the predetermined signal strength range threshold may be a range of RSSIs of 10dBm, 15dBm or other signal strength range. For example, external device 12 may refrain from transmitting the instruction to IMD 10 until external device 12 determines that the range of signal strengths for an idle communication link is less than the predetermined signal strength range threshold for the predetermined length of time, e.g., three seconds. For example, external device 12 may initiate a communication session with IMD 10 in response to receiving one or more advertisements for communication, but may wait until external device 12 determines that the range of signal strengths of the communication link between external device 12 and IMD 10 is less than the predetermined signal strength range threshold for the predetermined length of time before transmitting the instruction to IMD 10. In some examples, there may be a plurality of such predetermined signal strength range thresholds, each associated with a different predetermined data threshold or there may be a plurality of predetermined lengths of time. For example, external device 12 may require each of the determined signal strength ranges between a lowest and highest determined RSSIs are less than -5dBm for a data transmission greater than a first predetermined data threshold size and that each of the determined signal strength ranges between the lowest and highest determined RSSIs are less than -4dBm for a data transmission of greater than a second predetermined data threshold, wherein the second predetermined data threshold is greater than the first predetermined data threshold. Alternatively, or in addition, the predetermined time period (e.g., two seconds) for the first predetermined data threshold may be less than the predetermined time period (e.g., three seconds) for the second predetermined data threshold. In some examples, there may be any number of predetermined signal strength range thresholds and/or predetermined period of seconds associated with different predetermined data thresholds. This restriction may reduce the likelihood that the transmission of the data from IMD 10 to external device 12 is interrupted.

In some examples, the predetermined restriction includes a current time within a time frame that histogram data is indicative of a successful communication of data from IMD 10 to external device 12. For example, external device 12 may store data indicative of successful communication with IMD 10 and unsuccessful communications with IMD 10 over time. This data may be histogram data. External device 12 may compare a current time and/or day of the week with stored histogram data to determine whether the histogram data is indicative of a successful communication of data from IMD 10 to external device 12 in the past. For example, if the current time and day of the week is Saturday at 12pm, IMD 10 may determine whether past communications on Saturday at 12pm have been successful based on the histogram data. In some examples, external device 12 may look up several communications sessions from the histogram data, e.g., a predetermined number of communication sessions from Saturdays at 12pm and determine whether a predetermined number threshold of the predetermined number of communication sessions were successful. For example, external device 12 may look up the last ten communications sessions from the histogram data occurring on Saturdays at 12pm and determine whether seven of the last ten communication sessions were successful. This restriction may reduce the likelihood that the transmission of the data from IMD 10 to external device 12 is interrupted.

In some examples, the predetermined restriction includes predetermined conditional logic, such as if this then that (IFTTT) logic. For example, external device 12 and/or IMD 10 may be configured to incorporate IFTTT logic to improve the probability of a successful transmission therebetween. For example, external device 12 may prevent data transmissions of significant size when the IFTTT logic indicates a lower probability of completing the transmission. For example, in the case where external device 12 is a mobile device, if the expected amount of data to be transmitted is greater than 10 KBytes, external device 12 may only initiate a communication session with IMD 10 if external device 12 is at a home of patient 4, unless has been more than 12 hours since patient 4 was at home. For example, IMD 10 may use geo-fencing techniques to determine whether patient 4 is at home. In another example, external device 12 may be configured to postpone a communication session with IMD 10 if wireless internet bandwidth insufficient to support the communication session, for example, is less than a predetermined bandwidth threshold. In another example, external device 12 may be configured provide a notification to patient 4 asking patient 4 to sit by external device 12 until a follow-up notification is provided by external device 12, or to sit by external device for a predetermined period of time, e.g., ten minutes, when external device 12 wants to initiate a communication session with IMD 10. The notification may be an auditory, visual, or tactile notification, for example. Many other examples of the use of IFTTT logic may exist and still fall within the scope of this disclosure.

In some examples, the predetermined restriction includes a time at which patient 4 believes is a good time for communicating with IMD 10. For example, external device 12 may prompt patient 4 to provide a timeframe when patient 4 believes that IMD 10 and external device 12 may be in close proximity to each other and external device 12 may receive a response to the prompt from patient 4 through a user interface of external device 12. Alternatively, or in addition, external device 12 may prompt patient 4 to confirm that a present time is a good time for external device 12 to communicate with IMD 10 and external device 12 may receive a response to the prompt from patient 4 through a user interface of external device 12. In some examples, external device 12 may provide instructions to patient 4 on how to improve the likelihood of successful communication, such as hold the phone over their chest until the phone beeps, vibrates, or displays a message indicative of the existence of a communication session. For example, external device 12 may send the instructions and then when external device 12 establishes communications with IMD 10, external device 12 may audibly, haptically, or visually indicate to patient 4 that a communication session is being conducted.

In some examples, the predetermined restriction includes times of previous successful communications between IMD 10 and external device 12. For example, IMD 10 may store information indicative of prior successful communications with external device 12 and only transmit large payloads during times corresponding to prior successful communications.

In some examples, the predetermined restriction includes the transmission being relatively urgent. For example, if IMD 10 senses a cardiac event, such as cardiac arrest, a transmission regarding that cardiac event may be more urgent than a transmission regarding normal cardiac activity.

While the techniques of this disclosure are primarily described as being implemented by external device 12, in some examples the techniques of this disclosure may be implemented by IMD 10, another device, or any combination of such devices.

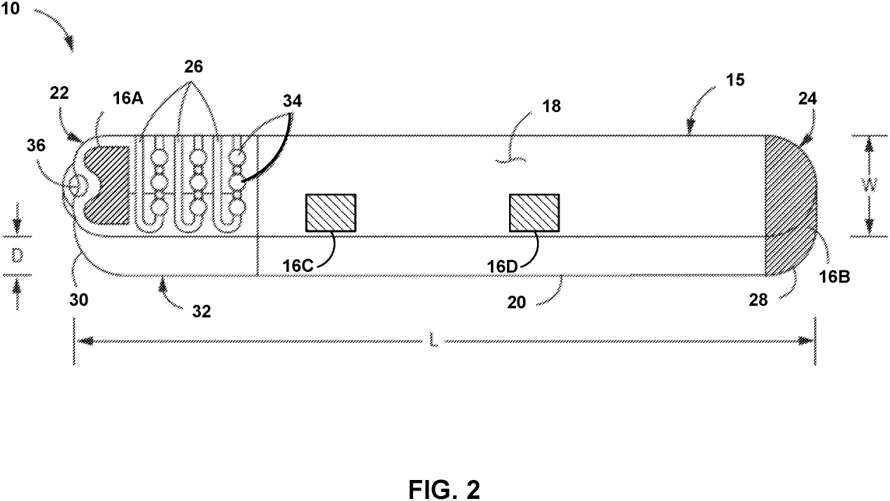

FIG. 2 is a conceptual drawing illustrating an example configuration of IMD 10 of the medical device system 2 of FIG. 1, in accordance with one or more techniques described herein. In the example shown in FIG. 2, IMD 10 may include a leadless, subcutaneously-implantable monitoring device having housing 15, proximal electrode 16A, and distal electrode 16B. Housing 15 may further include first major surface 18, second major surface 20, proximal end 22, and distal end 24. In some examples, IMD 10 may include one or more additional electrodes 16C, 16D positioned on one or both of major surfaces 18, 20 of IMD 10. Housing 15 encloses electronic circuitry located inside the IMD 10, and protects the circuitry contained therein from fluids such as body fluids. In some examples, electrical feedthroughs provide electrical connection of electrodes 16A-16D, and antenna 26, to circuitry within housing 15. In some examples, electrode 16B may be formed from an uninsulated portion of conductive housing 15.

In the example shown in FIG. 2, IMD 10 is defined by a length L, a width W, and thickness or depth D. In this example, IMD 10 is in the form of an elongated rectangular prism in which length L is significantly greater than width W, and in which width W is greater than depth D. However, other configurations of IMD 10 are contemplated, such as those in which the relative proportions of length L, width W, and depth D vary from those described and shown in FIG. 2. In some examples, the geometry of the IMD 10, such as the width W being greater than the depth D, may be selected to allow IMD 10 to be inserted under the skin of the patient using a minimally invasive procedure and to remain in the desired orientation during insertion. In addition, IMD 10 may include radial asymmetries (e.g., the rectangular shape) along a longitudinal axis of IMD 10, which may help maintain the device in a desired orientation following implantation.

In some examples, a spacing between proximal electrode 16A and distal electrode 16B may range from about 30-55 mm, about 35-55 mm, or about 40-55 mm, or more generally from about 25-60 mm. Overall, IMD 10 may have a length L of about 20-30 mm, about 40-60 mm, or about 45-60 mm. In some examples, the width W of major surface 18 may range from about 3-10 mm, and may be any single width or range of widths between about 3-10 mm. In some examples, a depth D of IMD 10 may range from about 2-9 mm. In other examples, the depth D of IMD 10 may range from about 2-5 mm, and may be any single or range of depths from about 2-9 mm. In any such examples, IMD 10 is sufficiently compact to be implanted within the subcutaneous space of patient 4 in the region of a pectoral muscle.

IMD 10, according to an example of the present disclosure, may have a geometry and size designed for ease of implant and patient comfort. Examples of IMD 10 described in this disclosure may have a volume of 3 cubic centimeters (cm³) or less, 1.5 cm³ or less, or any volume therebetween. In addition, in the example shown in FIG. 2, proximal end 22 and distal end 24 are rounded to reduce discomfort and irritation to surrounding tissue once implanted under the skin of patient 4.

In the example shown in FIG. 2, first major surface 18 of IMD 10 faces outward towards the skin, when IMD 10 is inserted within patient 4, whereas second major surface 20 is faces inward toward musculature of patient 4. Thus, first and second major surfaces 18, 20 may face in directions along a sagittal axis of patient 4 (see FIG. 1), and this orientation may be maintained upon implantation due to the dimensions of IMD 10.

Proximal electrode 16A and distal electrode 16B may be used to sense cardiac EGM signals (e.g., electrocardiogram (ECG) signals) when IMD 10 is implanted subcutaneously in patient 4. In some examples, processing circuitry of IMD 10 also may determine whether cardiac ECG signals of patient 4 are indicative of arrhythmia or other abnormalities, which processing circuitry of IMD 10 may evaluate in determining whether a medical condition (e.g., heart failure, sleep apnea, or COPD) of patient 4 has changed. The cardiac ECG signals may be stored in a memory of the IMD 10, and data derived from the cardiac ECG signals may be transmitted via integrated antenna 26 to another medical device, such as external device 12. In some examples, one or both of electrodes 16A and 16B also may be used by IMD 10 to detect impedance values during impedance measurements performed by IMD 10. In some examples, such impedance values detected by IMD 10 may reflect a resistance value associated with a contact between electrodes 16A, 16B, and target tissue of patient 4. Additionally, in some examples, electrodes 16A, 16B may be used by communication circuitry of IMD 10 for tissue conductance communication (TCC) communication with external device 12 or another device.

In the example shown in FIG. 2, proximal electrode 16A is in close proximity to proximal end 22, and distal electrode 16B is in close proximity to distal end 24 of IMD 10. In this example, distal electrode 16B is not limited to a flattened, outward facing surface, but may extend from first major surface 18, around rounded edges 28 or end surface 30, and onto the second major surface 20 in a three-dimensional curved configuration. As illustrated, proximal electrode 16A is located on first major surface 18 and is substantially flat and outward facing. However, in other examples not shown here, proximal electrode 16A and distal electrode 16B both may be configured like proximal electrode 16A shown in FIG. 2, or both may be configured like distal electrode 16B shown in FIG. 2. In some examples, additional electrodes 16C and 16D may be positioned on one or both of first major surface 18 and second major surface 20, such that a total of four electrodes are included on IMD 10. Any of electrodes 16A-16D may be formed of a biocompatible conductive material. For example, any of electrodes 16A-16D may be formed from any of stainless steel, titanium, platinum, iridium, or alloys thereof. In addition, electrodes of IMD 10 may be coated with a material such as titanium nitride or fractal titanium nitride, although other suitable materials and coatings for such electrodes may be used.

In the example shown in FIG. 2, proximal end 22 of IMD 10 includes header assembly 32 having one or more of proximal electrode 16A, integrated antenna 26, anti-migration projections 34, and suture hole 36. Integrated antenna 26 is located on the same major surface (e.g., first major surface 18) as proximal electrode 16A, and may be an integral part of header assembly 32. In other examples, integrated antenna 26 may be formed on the major surface opposite from proximal electrode 16A, or, in still other examples, may be incorporated within housing 15 of IMD 10. Antenna 26 may be configured to transmit or receive electromagnetic signals for communication. For example, antenna 26 may be configured to transmit to or receive signals from a programmer via inductive coupling, electromagnetic coupling, tissue conductance, Near Field Communication (NFC), Radio Frequency Identification (RFID), Bluetooth®, BLE, Wi-Fi®, or other proprietary or non-proprietary wireless telemetry communication schemes. Antenna 26 may be coupled to communication circuitry of IMD 10, which may drive antenna 26 to transmit signals to external device 12 and may transmit signals received from external device 12 to processing circuitry of IMD 10 via communication circuitry.

IMD 10 may include several features for retaining IMD 10 in position once subcutaneously implanted in patient 4. For example, as shown in FIG. 2, housing 15 may include anti-migration projections 34 positioned adjacent integrated antenna 26. Anti-migration projections 34 may include a plurality of bumps or protrusions extending away from first major surface 18 and may help prevent longitudinal movement of IMD 10 after implantation in patient 4. In other examples, anti-migration projections 34 may be located on the opposite major surface as proximal electrode 16A and/or integrated antenna 26. In addition, in the example shown in FIG. 2 header assembly 32 includes suture hole 36, which provides another means of securing IMD 10 to the patient to prevent movement following insertion. In the example shown, suture hole 36 is located adjacent to proximal electrode 16A. In some examples, header assembly 32 may include a molded header assembly made from a polymeric or plastic material, which may be integrated or separable from the main portion of IMD 10.

Electrodes 16A and 16B may be used to sense cardiac ECG signals, as described above. Additional electrodes 16C and 16D may be used to sense subcutaneous tissue impedance, in addition to or instead of electrodes 16A, 16B, in some examples. In some examples, processing circuitry of IMD 10 may determine an impedance value of patient 4 based on signals received from at least two of electrodes 16A-16D. For example, processing circuitry of IMD 10 may generate one of a current or voltage signal, deliver the signal via a selected two or more of electrodes 16A-16D, and measure the resulting other of current or voltage. Processing circuitry of IMD 10 may determine an impedance value based on the delivered current or voltage and the measured voltage or current.

In some examples, IMD 10 may include one or more additional sensors, such as one or more accelerometers (not shown) and/or one or more light sensors (not shown). Such accelerometers may be 3D accelerometers configured to generate signals indicative of one or more types of movement of the patient, such as gross body movement (e.g., motion) of the patient, patient posture, movements associated with the beating of the heart, or coughing, rales, or other respiration abnormalities. One or more of the parameters monitored by IMD 10 (e.g., impedance, EGM) may fluctuate in response to changes in one or more such types of movement. For example, changes in parameter values sometimes may be attributable to increased patient motion (e.g., exercise or other physical motion as compared to immobility) or to changes in patient posture, and not necessarily to changes in a medical condition. While IMD 10 is described as including various components, in some examples IMDs which may implement techniques of this disclosure may include other components, such as a therapy component that is configured to deliver therapy to patient 4, including, but not limited to a pulse generator for delivering electrical stimulation (e.g., pacing pulses, defibrillation shocks, etc.), a motor for providing left ventricle assist device (LVAD) therapy, or a drug pump and reservoir for delivering drugs to patient 4.

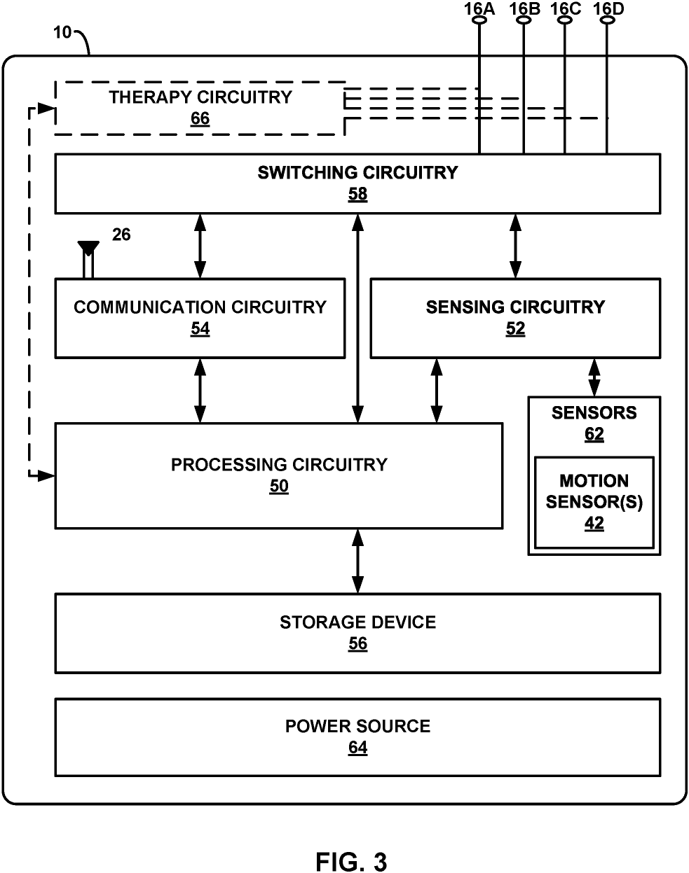

FIG. 3 is a functional block diagram illustrating an example configuration of IMD 10 of FIGS. 1 and 2, in accordance with one or more techniques described herein. In the illustrated example, IMD 10 includes electrodes 16, antenna 26, processing circuitry 50, sensing circuitry 52, communication circuitry 54, storage device 56, switching circuitry 58, sensors 62 including motion sensor(s) 42, and power source 64. Although not illustrated in FIG. 3, sensors 62 may include one or more light detectors.

Processing circuitry 50 may include fixed function circuitry and/or programmable processing circuitry. Processing circuitry 50 may include any one or more of a microprocessor, a controller, a DSP, an ASIC, an FPGA, or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof.

Sensing circuitry 52 and communication circuitry 54 may be selectively coupled to electrodes 16A-16D via switching circuitry 58, as controlled by processing circuitry 50. Sensing circuitry 52 may monitor signals from electrodes 16A-16D in order to monitor electrical activity of heart (e.g., to produce an EGM), and/or subcutaneous tissue impedance, the impedance being indicative of at least some aspects respiratory patterns of patient 4 and the EMG being indicative of at least some aspects cardiac patterns of patient 4. In some examples, a subcutaneous impedance signal collected by IMD 10 may indicate a respiratory rate and/or a respiratory intensity of patient 4 and an EMG collected by IMD 10 may indicate a heart rate of patient 4 and an atrial fibrillation (AF) burden of patient 4. Sensing circuitry 52 also may monitor signals from sensors 62, which may include motion sensor(s) 42, such as accelerometer(s), and any additional sensors, such as light detectors or pressure sensors, that may be positioned on IMD 10. In some examples, sensing circuitry 52 may include one or more filters and amplifiers for filtering and amplifying signals received from one or more of electrodes 16A-16D and/or motion sensor(s) 42.

Communication circuitry 54 may include any suitable hardware, firmware, software or any combination thereof for communicating with another device, such as external device 12 or another IMD or sensor, such as a pressure sensing device. Under the control of processing circuitry 50, communication circuitry 54 may receive downlink telemetry from, as well as transmit uplink telemetry to, external device 12 or another device with the aid of an internal or external antenna, e.g., antenna 26. In some examples, communication circuitry 54 may transmit advertisements for communication intended to be received by external device 12. Such advertisements may be regularly sent at predetermined intervals. In addition, processing circuitry 50 may communicate, via communication circuitry 54, with a networked computing device via an external device (e.g., external device 12) and a computer network, such as the Medtronic CareLink® Network developed by Medtronic, plc, of Dublin, Ireland.

A clinician, patient 4, or other user may retrieve data from IMD 10 using external device 12, or by using another local or networked computing device configured to communicate with processing circuitry 50 via communication circuitry 54. The clinician may also program parameters of IMD 10 using external device 12 or another local or networked computing device.

In some examples, storage device 56 includes computer-readable instructions that, when executed by processing circuitry 50, cause IMD 10 and processing circuitry 50 to perform various functions attributed to IMD 10 and processing circuitry 50 herein. Storage device 56 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media.

Power source 64 is configured to deliver operating power to the components of IMD 10. Power source 64 may include a battery and a power generation circuit to produce the operating power. In some examples, the battery is non-rechargeable. In some examples, the battery is rechargeable to allow extended operation. In some examples, recharging is accomplished through proximal inductive interaction between an external charger and an inductive charging coil within external device 12. Power source 64 may include any one or more of a plurality of different battery types, such as nickel cadmium batteries and lithium ion batteries. A non-rechargeable battery may be selected to last for several years, while a rechargeable battery may be inductively charged from an external device, e.g., on a daily or weekly basis.

In some examples, processing circuitry 50 of IMD 10 may use sensing circuitry 52 and/or sensors 62 (e.g., motion sensor 42) to determine a posture of patient 4 and/or a position of patient 4. Processing circuitry 50 IMD 10 may use that determination to determine whether patient 4 is relatively stationary or a likelihood that IMD 10 is within communication range of external device 12. Processing circuitry 50 may include information indicative of how stationary patient 4 may be and/or how likely that IMD 10 is within communication range of external device 12 in an advertisement for communication. External device 12 may use such information to determine whether to connect with IMD 10.

In some examples, IMD 10 may optionally include therapy delivery circuitry 66 (shown in dashed lines). Therapy delivery circuitry may include a pulse generator for delivering electrical stimulation (e.g., pacing pulses, defibrillation shocks, etc.), a motor for providing left ventricle assist device (LVAD) therapy, a drug pump and reservoir for delivering drugs to patient 4, or any other circuitry configured to deliver therapy to patient 4. In some examples, therapy circuitry 66 may be configured to deliver therapy through electrodes 16A-16D or through other electrodes (not shown).

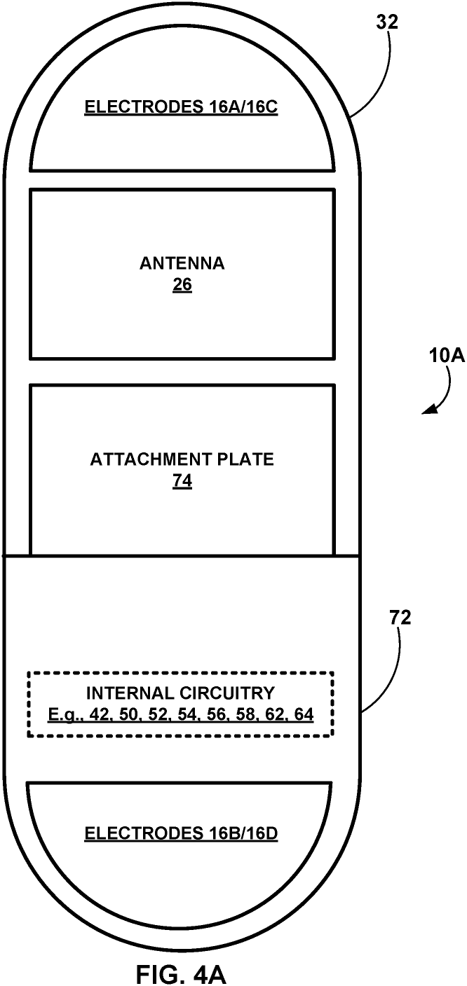

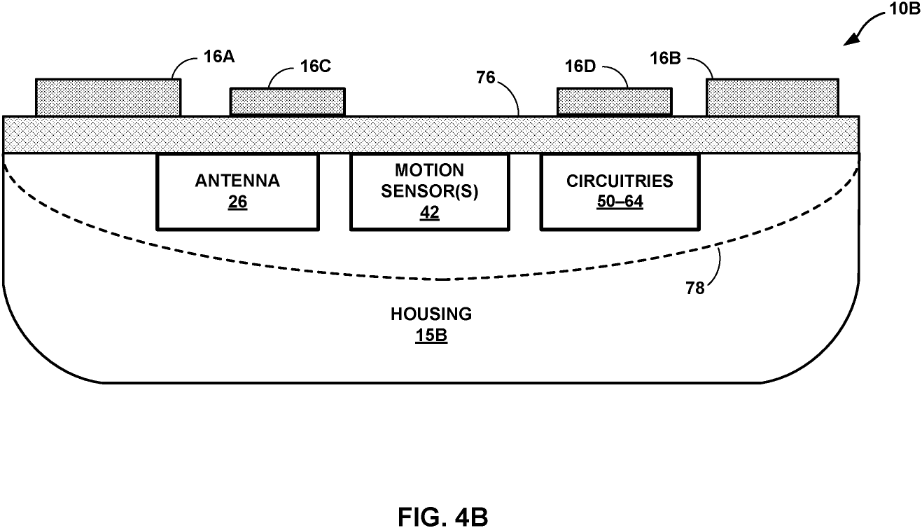

FIGS. 4A and 4B illustrate two additional example IMDs that may be substantially similar to IMD 10 of FIGS. 1-3, but which may include one or more additional features, in accordance with one or more techniques described herein. The components of FIGS. 4A and 4B may not necessarily be drawn to scale, but instead may be enlarged to show detail. FIG. 4A is a block diagram of a top view of an example configuration of an IMD 10A. FIG. 4B is a block diagram of a side view of example IMD 10B, which may include an insulative layer as described below.

FIG. 4A is a conceptual drawing illustrating another example IMD 10A that may be substantially similar to IMD 10 of FIG. 1. In addition to the components illustrated in FIGS. 1-3, the example of IMD 10 illustrated in FIG. 4A also may include a body portion 72 and an attachment plate 74. Attachment plate 74 may be configured to mechanically couple header assembly 32 to body portion 72 of IMD 10A. Body portion 72 of IMD 10A may be configured to house one or more of the internal components of IMD 10 illustrated in FIG. 3, such as one or more of processing circuitry 50, sensing circuitry 52, communication circuitry 54, storage device 56, switching circuitry 58, internal components of sensors 62, and power source 64. In some examples, body portion 72 may be formed of one or more of titanium, ceramic, or any other suitable biocompatible materials.

FIG. 4B is a conceptual drawing illustrating another example IMD 10B that may include components substantially similar to IMD 10 of FIG. 1. In addition to the components illustrated in FIGS. 1-3, the example of IMD 10B illustrated in FIG. 4B also may include a wafer-scale insulative cover 76, which may help insulate electrical signals passing between electrodes 16A-16D and processing circuitry 50. In some examples, insulative cover 76 may be positioned over an open housing 15B to form the housing for the components of IMD 10B. One or more components of IMD 10B (e.g., antenna 26, light emitter 38, processing circuitry 50, sensing circuitry 52, communication circuitry 54, switching circuitry 58, and/or power source 64) may be formed on a bottom side of insulative cover 76, such as by using flip-chip technology. Insulative cover 76 may be flipped onto a housing 15B. When flipped and placed onto housing 15B, the components of IMD 10B formed on the bottom side of insulative cover 76 may be positioned in a gap 78 defined by housing 15B.

Insulative cover 76 may be configured so as not to interfere with the operation of IMD 10B. For example, one or more of electrodes 16A-16D may be formed or placed above or on top of insulative cover 76, and electrically connected to switching circuitry 58 through one or more vias (not shown) formed through insulative cover 76. Insulative cover 76 may be formed of sapphire (i.e., corundum), glass, parylene, and/or any other suitable insulating material.

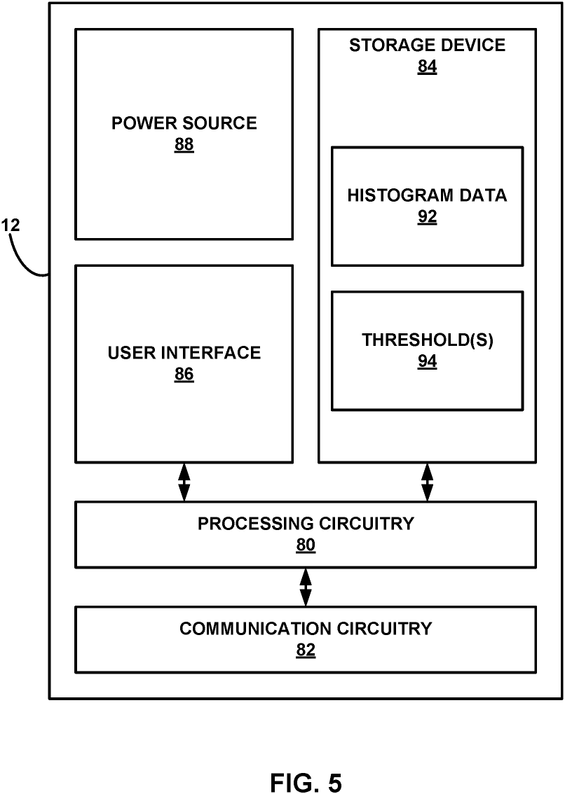

FIG. 5 is a block diagram illustrating an example configuration of components of external device 12, in accordance with one or more techniques of this disclosure. In the example of FIG. 5, external device 12 includes processing circuitry 80, communication circuitry 82, storage device 84, user interface 86, power source 88, and sensors 90. In some examples, external device 12 is a mobile device. In some examples, external device 12 is a stationary device.

Processing circuitry 80, in one example, may include one or more processors that are configured to implement functionality and/or process instructions for execution within external device 12. For example, processing circuitry 80 may be capable of processing instructions stored in storage device 84. Processing circuitry 80 may include, for example, microprocessors, DSPs, ASICs, FPGAs, or equivalent discrete or integrated logic circuitry, or a combination of any of the foregoing devices or circuitry. Accordingly, processing circuitry 80 may include any suitable structure, whether in hardware, software, firmware, or any combination thereof, to perform the functions ascribed herein to processing circuitry 80. Processing circuitry 80 may be configured to determine an expected amount of data to be transmitted by IMD 10 to external device 12 as described in detail above. Processing circuitry 80 may be configured to determine that an expected amount of data to be transmitted by IMD 10 to external device 12 is greater than or equal to a predetermined data threshold. Processing circuitry 80 may be configured to, based on the expected amount of data to be transmitted being greater than or equal to the predetermined data threshold, determine that a predetermined restriction is met. Processing circuitry 80 may, based on the predetermined restriction being met, control communication circuitry 82 to transmit an instruction to the implanted medical device. The instruction may include an instruction for IMD 10 to transmit the data to be transmitted to external device 12.

Communication circuitry 82 may include any suitable hardware, firmware, software or any combination thereof for communicating with another device, such as IMD 10. Under the control of processing circuitry 80, communication circuitry 82 may receive downlink telemetry from, as well as transmit uplink telemetry to, IMD 10, or another device. For example, communication circuitry 82 may be configured to sense an advertisement for communication from communication circuitry 54 (FIG. 3) of IMD 10 at an interval known to external device 12. Communication circuitry 82 may also be configured to sense a beacon from, for example, a wireless access point, which may be associated with a geo-location. The geo-location of external device 12 may be used with IFTTT logic as a restriction on communications between IMD 10 and external device 12, as discussed above with respect to FIG. 1.

Storage device 84 may be configured to store information within external device 12 during operation. Storage device 84 may include a computer-readable storage medium or computer-readable storage device. In some examples, storage device 84 includes one or more of a short-term memory or a long-term memory. Storage device 84 may include, for example, RAM, dynamic random access memories (DRAM), static random access memories (SRAM), magnetic discs, optical discs, flash memories, or forms of electrically programmable memories (EPROM) or EEPROM. In some examples, storage device 84 is used to store data indicative of instructions for execution by processing circuitry 80. Storage device 84 may be used by software or applications running on external device 12 to temporarily store information during program execution.

Storage device 84 may store histogram data 92 which may be used by processing circuitry 80 to determine whether histogram data 92 is indicative of a successful communication of data from IMD 10 to external device 12 during a timeframe associated with a current time. Storage device 84 may also store threshold(s) 94 which may be used by processing circuitry 80 when determining whether a predetermined restriction is met. For example, threshold(s) 94 may include predetermined data threshold(s), predetermined signal strength threshold(s), predetermined signal strength range threshold(s), predetermined bandwidth threshold(s), or other thresholds (which may be associated with the predetermined restriction).

Data exchanged between external device 12 and IMD 10 may include operational parameters. External device 12 may transmit data including computer readable instructions which, when implemented by IMD 10, may control IMD 10 to change one or more operational parameters and/or export collected data. For example, processing circuitry 80 may transmit an instruction to IMD 10, via communication circuitry82, which requests IMD 10 to export collected data (e.g., sensed data by sensor(s) 62 or sensing circuitry 52) to external device 12. In turn, external device 12 may receive the collected data from IMD 10 and store the collected data in storage device 84. Additionally, or alternatively, processing circuitry 80 may export instructions to IMD 10 requesting IMD 10 to update electrode combinations for stimulation or sensing.

A user, such as a clinician or patient 4, may interact with external device 12 through user interface 86. User interface 86 includes a display (not shown), such as an LCD or LED display or other type of screen, with which processing circuitry 80 may present information related to IMD 10 (e.g., EGM signals obtained from at least one electrode or at least one electrode combination). In addition, user interface 86 may include an input mechanism to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 80 of external device 12 and provide input. In other examples, user interface 86 also includes audio circuitry for providing audible notifications, instructions or other sounds to patient 4, receiving voice commands from patient 4, or both. Storage device 84 may include instructions for operating user interface 86 and for managing power source 88.

Power source 88 is configured to deliver operating power to the components of external device 12. Power source 88 may include a battery and a power generation circuit to produce the operating power. In some examples, the battery is rechargeable to allow extended operation. Recharging may be accomplished by electrically coupling power source 88 to a cradle or plug that is connected to an alternating current (AC) outlet. In addition, recharging may be accomplished through proximal inductive interaction between an external charger and an inductive charging coil within external device 12. In other examples, traditional batteries (e.g., nickel cadmium or lithium ion batteries) may be used. In addition, external device 12 may be directly coupled to an alternating current outlet to operate.

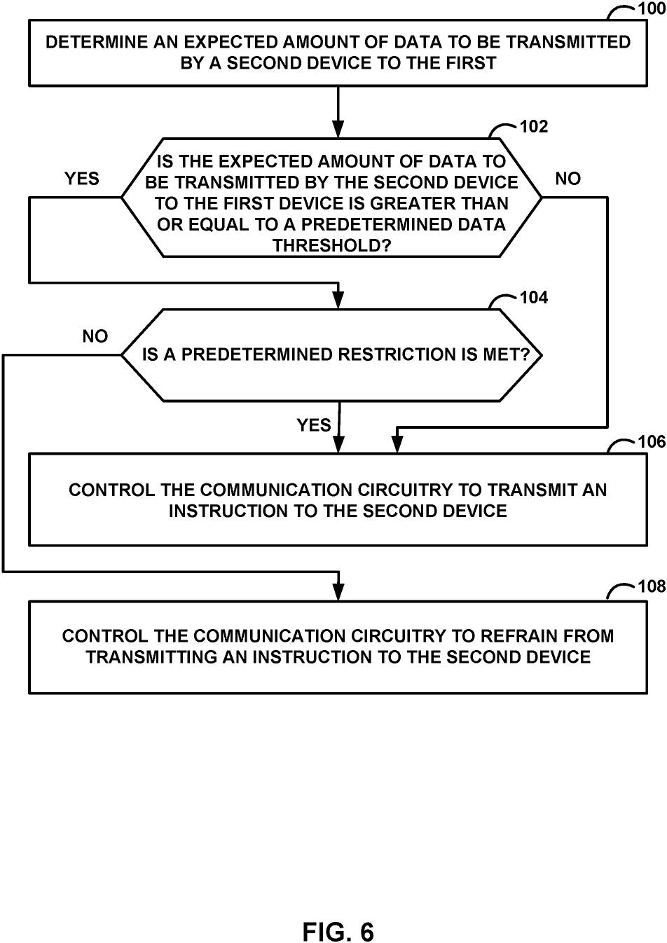

FIG. 6 is a flow diagram illustrating an example operation for improving power consumption of an IMD, in accordance with one or more techniques of this disclosure. FIG. 6 is described with respect to IMD 10, and external device 12 of FIGS. 1-5. However, the techniques of FIG. 6 may be performed by different components of IMD 10, or external device 12, or by additional or alternative devices or device systems.

A first device (e.g., external device 12) may determine that an expected amount of data to be transmitted by a second device (e.g., IMD 10) to a first device (e.g., external device 12) (100). For example, processing circuitry 80 may analyze the type of instruction that external device 12 is going to transmit to IMD 10, the last time IMD 10 transmitted stored data to external device 12, and/or sizes of historical data transmissions from IMD 10 to external device 12, to determine the expected amount of data to be transmitted by IMD 10 to external device 12. For example, if patient 4 input a command to download all sensed physiological data stored in storage device 56, and it has been one day since the last time IMD 10 transmitted data to external device 12, the expected amount of data to be transmitted by IMD 10 to external device 12 may be relatively large.

The first device may determine whether the expected amount of data to be transmitted by the second device to the first device is greater than or equal to a predetermined data threshold (102). For example, processing circuitry 80 may compare the expected amount of data to be transmitted by IMD 10 to the predetermined data threshold which may be stored in threshold(s) 94 of storage device 84 to determine whether the amount of data to be transmitted is greater than or equal to the predetermined data threshold.

If the first device determines that the expected amount of data to be transmitted by the second device to the first device is not greater than or equal to the predetermined data threshold (the “NO” path from box 102), processing circuitry of the first device may control communication circuitry to transmit an instruction to the second device (e.g., IMD 10) (106). For example, processing circuitry 80 may not check to determine whether a predetermined restriction is met and may proceed to control communication circuitry 82 to transmit the instruction to IMD 10.

If the first device determines that the expected amount of data to be transmitted by the second device to the first device is greater than or equal to the predetermined data threshold (the “YES” path from box 102), the first device may determine whether a predetermined restriction is met (104).

For example, processing circuitry 80 may determine whether the predetermined restriction is met prior to external device 12 transmitting an instruction to IMD 10. In some examples, the predetermined restriction includes the first device (e.g., external device 12) discovering an advertisement for communication from the second device (e.g., IMD 10) in a predetermined number of consecutive advertising intervals. In some examples, the predetermined restriction includes the first device discovering an advertisement for communication from the second device in a first predetermined number of advertising intervals out of a second predetermined number of consecutive advertising intervals. In some examples, the predetermined restriction includes the first device discovering a first predetermined number of advertisements for communication from the second device during a predetermined period of time. In some examples, the predetermined restriction includes a signal strength of an advertisement for communication from the second device being greater than or equal to a signal strength threshold. In some examples, the predetermined restriction includes a signal strength of an idle communication link between the implantable medical device and the external device being greater than or equal to a signal strength threshold for a predetermined length of time. In some examples, the predetermined restriction includes a range of signal strengths of an idle communication link between the implantable medical device and the external device being smaller than to a signal strength range threshold for a predetermined length of time. In some examples, the predetermined restriction includes that histogram data is indicative of a successful communication of data from the implantable medical device to the external device during a time frame associated with a current time. In some examples, the predetermined restriction includes predetermined conditional logic, such as IFTTT logic. In some examples, the predetermined restriction includes a time at which patient 4 believes is a good time for communicating with IMD 10. In some examples, the predetermined restriction comprises an urgency level of a transmission of the data to be transmitted by the second device to the first device.

If the first device determines that the predetermined restriction is met (the “YES” path from box 104), the first device, based on the predetermined restriction being met, control communication circuitry to transmit an instruction to the second device (106). For example, based on the predetermined restriction being met, processing circuitry 80 may control communication circuitry 82 to transmit an instruction to IMD 10 for IMD 10 to transmit the data to be transmitted (e.g., sensed physiological parameters of patient 4) to external device 12. In some examples, processing circuitry 80 may control communication circuitry 82 to transmit the instruction after a predetermined length of time.

If the first device determines that the predetermined restriction is not met (the “NO” path from box 104), the first device may control communication circuitry to refrain from transmitting an instruction to the second device. For example, processing circuitry 80 may determine that any response by IMD 10 to the instruction may be likely to be unsuccessful and may control communication circuitry 82 to refrain from transmitting the instruction. In some examples, processing circuitry 80 may control communication circuitry 82 to transmit a message to IMD 10 to increase the time between advertising intervals (hereinafter referred to as an “advertising interval message”). For example, external device 12 may transmit the advertising interval message to IMD 10 to increase the time between advertising intervals from every 3 minutes to every 15 minutes. In this manner, IMD 10 may save battery charge by not advertising for communication as often as IMD 10 otherwise would. External device 12 may later transmit another message to IMD 10 to return to the original predetermined advertising intervals, for example, when the likelihood of successful communication is relatively higher.

In some examples, processing circuitry 80 may return to box 104 or may wait for a period of time or for someone to enter a new instruction into user interface 86 and return to box 100 or box 104.

By placing a restriction(s) on when data is transmitted by IMD 10 to external device 12 a communication session between external device 12 and IMD 10 may be more likely to be successful and therefore the battery life of IMD 10 may be extended, as IMD 10 may avoid repeated transmission of the same data. In the case where the battery of IMD 10 is non-rechargeable, this may lengthen the life of IMD 10 and extend the time before patient 4 undergoes replacement surgery. In the case where the battery of IMD 10 is rechargeable, this may lengthen the recharge interval which may be beneficial to patient 4 as it may offer patient 4 more flexibility in daily living. Additionally, the techniques of this disclosure may improve the reliability of connections between IMD 10 and external device 12, the predictability of such connections, and/or the speed of transfer of information over such connections.

The techniques described in this disclosure may be implemented, at least in part, in hardware, software, firmware, or any combination thereof. For example, various aspects of the techniques may be implemented within one or more microprocessors, DSPs, ASICs, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

This disclosure includes the following non-limiting examples.

Example 1. A first device comprising: communication circuitry configured to communicate with a second device; and processing circuitry configured to: determine an expected amount of data to be transmitted by the second device to the first device; determine that the expected amount of data to be transmitted by the second device to the first device is greater than or equal to a predetermined data threshold; based on the expected amount of data to be transmitted being greater than or equal to the predetermined data threshold, determine that a predetermined restriction is met; and based on the predetermined restriction being met, control the communication circuitry to transmit an instruction to the second device.

Example 2. The first device of claim 1, wherein the instruction comprises an instruction for the second device to transmit the data to be transmitted to the first device.

Example 3. The first device of example 1 or example 2, wherein the predetermined restriction comprises the first device discovering an advertisement for communication from the second device in a predetermined number of consecutive advertising intervals.

Example 4. The first device of any of examples 1-3, wherein the predetermined restriction comprises the first device discovering an advertisement for communication from the second device in a first predetermined number of advertising intervals out of a second predetermined number of consecutive advertising intervals.

Example 5. The first device of any of examples 1-3, wherein the predetermined restriction comprises the first device discovering a first predetermined number of advertisements for communication from the second device during a predetermined period of time.

Example 6. The first device of any of examples 1-3, wherein the predetermined restriction comprises a signal strength of an advertisement for communication from the second device being greater than or equal to a predetermined signal strength threshold.

Example 7. The first device of any of examples 1-3, wherein the predetermined restriction comprises a signal strength of an idle communication link between the second device and the first device being greater than or equal to a predetermined signal strength threshold for a predetermined length of time and wherein the processing circuitry is configured to control the communication circuitry to transmit the instruction after the predetermined length of time.

Example 8. The first device of any of examples 1-3, wherein the predetermined restriction comprises a range of signal strengths of an idle communication link between the second device and the first device being smaller than to a predetermined signal strength range threshold for a predetermined length of time and wherein the processing circuitry is configured to control the communication circuitry to transmit the instruction after the predetermined length of time.

Example 9. The first device of any of examples 1-3, wherein the predetermined restriction comprises histogram data is indicative of a successful communication of data from the second device to the first device during a time frame associated with a current time.

Example 10. The first device of any of examples 1-3, wherein the predetermined restriction comprises predetermined conditional logic comprising if this then that logic.

Example 11. The first device of claim 1, wherein the predetermined restriction comprises an urgency level of a transmission of the data to be transmitted by the second device to the first device.

Example 12. The first device of any of examples 1-11, wherein the expected amount of data to be transmitted is a first expected amount of data to be transmitted and wherein the instruction is a first instruction, and wherein the processing circuitry is further configured to: determine a second expected amount of data to be transmitted by the second device to the first device; determine that the second expected amount of data to be transmitted by the second device to the first device is less than the predetermined data threshold; and based on the second expected amount of data to be transmitted being less than the predetermined data threshold, control the communication circuitry to transmit a second instruction to the second device.

Example 13. The first device of any of examples 1-12, wherein the expected amount of data to be transmitted is a first expected amount of data to be transmitted and wherein the instruction is a first instruction, and wherein the processing circuitry is further configured to: determine a second expected amount of data to be transmitted by the second device to the first device; determine that the second expected amount of data to be transmitted by the second device to the first device is greater than or equal to a predetermined data threshold; based on the second expected amount of data to be transmitted being greater than or equal to the predetermined data threshold, determine that the predetermined restriction is not met for the second expected amount of data; and based on the predetermined restriction being met for the second expected amount of data, control the communication circuitry to refrain from transmitting a second instruction to the second device.

Example 14. The first device of any of examples 1-13, wherein the processing circuitry is further configured to: determine that a battery charge level is below a predetermined charge threshold prior to determining the expected amount of data to be transmitted by the second device to the first device.

Example 15. A method comprising: determining, by processing circuitry of a first device, an expected amount of data to be transmitted by a second device to the first device; determining, by the processing circuitry, that the expected amount of data to be transmitted by the second device to the first device is greater than or equal to a predetermined data threshold; determining, by the processing circuitry and based on the expected amount of data to be transmitted being greater than or equal to the predetermined data threshold, that a predetermined restriction is met; and controlling communication circuitry, by the processing circuitry and based on the predetermined restriction being met, to transmit an instruction to the second device.

Example 16. The method of example 15, wherein the instruction comprises an instruction for the second device to transmit the data to be transmitted to the first device.

Example 17. The method of example 15 or example 16, wherein the predetermined restriction comprises the first device discovering an advertisement for communication from the second device in a predetermined number of consecutive advertising intervals.

Example 18. The method of any of examples 15-17, wherein the predetermined restriction comprises the first device discovering an advertisement for communication from the second device in a first predetermined number of advertising intervals out of a second predetermined number of consecutive advertising intervals.

Example 19. The method of any of examples 15-17, wherein the predetermined restriction comprises the first device discovering a first predetermined number of advertisements for communication from the second device during a predetermined period of time.

Example 20. The method of any of examples 15-17, wherein the predetermined restriction comprises a signal strength of an advertisement for communication from the second device being greater than or equal to a predetermined signal strength threshold.

Example 21. The method of any of examples 15-17, wherein the predetermined restriction comprises a signal strength of an idle communication link between the second device and the first device being greater than or equal to a predetermined signal strength threshold for a predetermined length of time and wherein the controlling the communication circuitry to transmit an instruction comprises controlling the communication circuitry to transmit the instruction after the predetermined length of time.

Example 22. The method of any of examples 15-17, wherein the predetermined restriction comprises a range of signal strengths of an idle communication link between the second device and the first device being smaller than to a predetermined signal strength range threshold for a predetermined length of time and wherein the controlling the communication circuitry to transmit an instruction comprises controlling the communication circuitry to transmit the instruction after the predetermined length of time.

Example 23. The method of any of examples 15-17, wherein the predetermined restriction comprises histogram data is indicative of a successful communication of data from the second device to the first device during a time frame associated with a current time.

Example 24. The method of any of examples 15-17, wherein the predetermined restriction comprises predetermined conditional logic.

Example 25. The method of any of examples 15-24, wherein the expected amount of data to be transmitted is a first expected amount of data to be transmitted and wherein the instruction is a first instruction, and wherein the method further comprises: determining, by the processing circuitry, a second expected amount of data to be transmitted by the second device to the first device; determining, by the processing circuitry, that the second expected amount of data to be transmitted by the second device to the first device is less than the predetermined data threshold; and controlling, by the processing circuitry and based on the second expected amount of data to be transmitted being less than the predetermined data threshold, the communication circuitry to transmit a second instruction to the second device.

Example 26. The method of any of examples 15-24, wherein the expected amount of data to be transmitted is a first expected amount of data to be transmitted and wherein the instruction is a first instruction, and wherein the method further comprises: determining, by the processing circuitry, a second expected amount of data to be transmitted by the second device to the first device; determining, by the processing circuitry, that the second expected amount of data to be transmitted by the second device to the first device is greater than or equal to a predetermined data threshold; determining, by the processing circuitry and based on the second expected amount of data to be transmitted being greater than or equal to the predetermined data threshold, that the predetermined restriction is not met for the second expected amount of data; and controlling, by the processing circuitry and based on the predetermined restriction being met for the second expected amount of data, the communication circuitry to refrain from transmitting a second instruction to the second device.

Example 27. The method of any of examples 15-26, wherein the processing circuitry is further configured to: determine that a battery charge level is below a predetermined charge threshold prior to determining the expected amount of data to be transmitted by the second device to the first device.

Example 28. A non-transitory computer-readable medium comprising instructions for causing one or more processors to: determine an expected amount of data to be transmitted by a second device to a first device; determine that the expected amount of data to be transmitted by the second device to the first device is greater than or equal to a predetermined data threshold; determine, based on the expected amount of data to be transmitted being greater than or equal to the predetermined data threshold, that a predetermined restriction is met; and control communication circuitry, based on the predetermined restriction being met, to transmit an instruction to the second device.

Example 29. The first device of any of examples 1-14, wherein the processing circuitry is further configured to: determine that a communication with the second device is likely to be successful; and based on the determination that the communication with the second device is likely to be successful, prompt a patient to remain in communication range of the first device until the communication is complete.

Example 30. The method of any of examples 15-27, further comprising determining that a communication with the second device is likely to be successful; and based on the determination that the communication with the second device is likely to be successful, prompting a patient to remain in communication range of the first device until the communication is complete.

Various aspects of the disclosure have been described. These and other aspects are within the scope of the following claims.

What is claimed is:

1. A first device comprising:
communication circuitry configured to communicate with a second device; and
processing circuitry configured to:
determine an expected amount of data to be transmitted by the second device to the first device;
determine that the expected amount of data to be transmitted by the second device to the first device is greater than or equal to a predetermined data threshold;
based on the expected amount of data to be transmitted being greater than or equal to the predetermined data threshold, determine that a predetermined restriction is met; and
based on the predetermined restriction being met, control the communication circuitry to transmit an instruction to the second device.

2. The first device of claim 1, wherein the instruction comprises an instruction for the second device to transmit the data to be transmitted to the first device.

3. The first device of claim 1, wherein the predetermined restriction comprises the first device discovering an advertisement for communication from the second device in a predetermined number of consecutive advertising intervals.

4. The first device of claim 1, wherein the predetermined restriction comprises the first device discovering an advertisement for communication from the second device in a first predetermined number of advertising intervals out of a second predetermined number of consecutive advertising intervals.

5. The first device of claim 1, wherein the predetermined restriction comprises the first device discovering a first predetermined number of advertisements for communication from the second device during a predetermined period of time.

6. The first device of claim 1, wherein the predetermined restriction comprises a signal strength of an advertisement for communication from the second device being greater than or equal to a predetermined signal strength threshold.

7. The first device of claim 1, wherein the predetermined restriction comprises a signal strength of an idle communication link between the second device and the first device being greater than or equal to a predetermined signal strength threshold for a predetermined length of time and wherein the processing circuitry is configured to control the communication circuitry to transmit the instruction after the predetermined length of time.

8. The first device of claim 1, wherein the predetermined restriction comprises a range of signal strengths of an idle communication link between the second device and the first device being smaller than to a predetermined signal strength range threshold for a predetermined length of time and wherein the processing circuitry is configured to control the communication circuitry to transmit the instruction after the predetermined length of time.

9. The first device of claim 1, wherein the predetermined restriction comprises histogram data is indicative of a successful communication of data from the second device to the first device during a time frame associated with a current time.

10. The first device of claim 1, wherein the predetermined restriction comprises predetermined conditional logic comprising if this then that logic.

11. The first device of claim 1, wherein the predetermined restriction comprises an urgency level of a transmission of the data to be transmitted by the second device to the first device.

12. The first device of claim 1, wherein the expected amount of data to be transmitted is a first expected amount of data to be transmitted and wherein the instruction is a first instruction, and wherein the processing circuitry is further configured to:
determine a second expected amount of data to be transmitted by the second device to the first device;
determine that the second expected amount of data to be transmitted by the second device to the first device is less than the predetermined data threshold; and
based on the second expected amount of data to be transmitted being less than the predetermined data threshold, control the communication circuitry to transmit a second instruction to the second device.

13. The first device of claim 1, wherein the expected amount of data to be transmitted is a first expected amount of data to be transmitted and wherein the instruction is a first instruction, and wherein the processing circuitry is further configured to:
determine a second expected amount of data to be transmitted by the second device to the first device;
determine that the second expected amount of data to be transmitted by the second device to the first device is greater than or equal to a predetermined data threshold;
based on the second expected amount of data to be transmitted being greater than or equal to the predetermined data threshold, determine that the predetermined restriction is not met for the second expected amount of data; and
based on the predetermined restriction being met for the second expected amount of data, control the communication circuitry to refrain from transmitting a second instruction to the second device.

14. The first device of claim 1, wherein the processing circuitry is further configured to determine that a battery charge level is below a predetermined charge threshold prior to determining the expected amount of data to be transmitted by the second device to the first device.

15. A method comprising:
determining, by processing circuitry of a first device, an expected amount of data to be transmitted by a second device to the first device;
determining, by the processing circuitry, that the expected amount of data to be transmitted by the second device to the first device is greater than or equal to a predetermined data threshold;
determining, by the processing circuitry and based on the expected amount of data to be transmitted being greater than or equal to the predetermined data threshold, that a predetermined restriction is met; and
controlling communication circuitry, by the processing circuitry and based on the predetermined restriction being met, to transmit an instruction to the second device.

16. The method of claim 15, wherein the instruction comprises an instruction for the second device to transmit the data to be transmitted to the first device.

17. The method of claim 15, wherein the expected amount of data to be transmitted is a first expected amount of data to be transmitted and wherein the instruction is a first instruction, and wherein the method further comprises:
determining, by the processing circuitry, a second expected amount of data to be transmitted by the second device to the first device;
determining, by the processing circuitry, that the second expected amount of data to be transmitted by the second device to the first device is less than the predetermined data threshold; and
controlling, by the processing circuitry and based on the second expected amount of data to be transmitted being less than the predetermined data threshold, the communication circuitry to transmit a second instruction to the second device.

18. The method of claim 15, wherein the expected amount of data to be transmitted is a first expected amount of data to be transmitted and wherein the instruction is a first instruction, and wherein the method further comprises:
determining, by the processing circuitry, a second expected amount of data to be transmitted by the second device to the first device;
determining, by the processing circuitry, that the second expected amount of data to be transmitted by the second device to the first device is greater than or equal to a predetermined data threshold;
determining, by the processing circuitry and based on the second expected amount of data to be transmitted being greater than or equal to the predetermined data threshold, that the predetermined restriction is not met for the second expected amount of data; and
controlling, by the processing circuitry and based on the predetermined restriction being met for the second expected amount of data, the communication circuitry to refrain from transmitting a second instruction to the second device.

19. The method of claim 15, wherein the processing circuitry is further configured to determine that a battery charge level is below a predetermined charge threshold prior to determining the expected amount of data to be transmitted by the second device to the first device.

20. A non-transitory computer-readable medium comprising instructions for causing one or more processors to:
determine an expected amount of data to be transmitted by a second device to a first device;
determine that the expected amount of data to be transmitted by the second device to the first device is greater than or equal to a predetermined data threshold;
determine, based on the expected amount of data to be transmitted being greater than or equal to the predetermined data threshold, that a predetermined restriction is met; and
control communication circuitry, based on the predetermined restriction being met, to transmit an instruction to the second device.
